(19) United States
(12) Patent Application Publication
(10) Pub. No.: US 2019/0374735 A1
(43) Pub. Date: Dec. 12, 2019

(54) Collar for Securing a Tracheostomy Tube to a Patient Using an Adhesive Patch and Method of Using the Collar

(71) Applicant: Pepper Medical Inc., West Chester, PA (US)

(72) Inventor: Gregory E. Doll, Bethesda, MD (US)

(21) Appl. No.: 16/437,054

(22) Filed: Jun. 11, 2019

Related U.S. Application Data
(60) Provisional application No. 62/683,231, filed on Jun. 11, 2018.

(51) Int. Cl. A61M 16/04

(52) U.S. Cl. CPC A61M 16/0497; A61M 2209/088; A61M 16/0465

(57) ABSTRACT

Provided is a collar for securing a tracheostomy tube to a patient. The collar has opposing neck connectors having a silicon pads to reversibly stick the color to a patient's neck, without encircling the patient's neck. Tabs on the collar are used to fasten the neckflange of the tracheostomy tube to the collar. Hook flaps are used to lock the tabs in place so that the neckflange cannot be easily removed from the collar by the patient.

### FIELD OF THE INVENTION

The invention relates to a collar for securing a tracheostomy tube to a patient and a method of using the collar to secure a tracheostomy tube to a patient.

### BACKGROUND OF THE INVENTION

A tracheotomy is a surgical procedure that includes making an incision on the anterior aspect of the neck and opening an airway through an incision in the trachea. The resulting stoma can serve independently as an airway or as a site for a tracheostomy tube (“trach tube”) to be inserted. The trach tube allows a patient to breathe without the use of his or her nose or mouth.

The trach tube generally consists of a curved tube that holds the stoma open. A neckflange or neck plate extends from the tube and has openings formed within it, Cloth ties or straps are generally attached to the openings in the neckflange of the trach tube and around the neck of the patient to hold the trach tube in place. The neckflange of the trach tube will often irritate and rub the skin on the anterior portion of the neck and the ties or straps will roll or slide causing the tie or strap to irritate the skin around the neck. As such, the ties or straps will often cause tissue breakdown, skin wounds, or irritation to the patient's neck.

Although patches have been in use, their primary use has been for wound coverage and not tube securement as in this application. While hook and loop has been in use, there have been disconnect problems with the hook and loop becoming disengaged due to patient intervention, clinician misuse, fluid and secretions on the product possibly leading to tube decannulation.

These dressings and collars and their designs suffer from several drawbacks. First, removal of the traditional dressing increases the danger that the scab or skin covering the wound will be removed along with the dressing. Another drawback is that many dressings fail to adequately aerate the wound.

In such instances, the healing of the wound is much slower. Another drawback is that traditional collars and holders wrap completely around the patient's neck, potentially leading to skin irritation, skin breakdown and skin infections. Another drawback is that these devices do not pad or support the skin near the tracheostomy stoma under the trach tube neckflange. Another drawback of these devices are that the Velcro® or hook and loop tabs can become disengaged thereby allowing the tracheostomy tube to become dislodged or decannulated leading to patient harm or death.

Other drawbacks to the traditional trach collar/holder which wraps around the patients neck to secure the tube is that movement of these ties create a substantial risk of infection to patients having undergone recent surgery to the head and/or neck. Moreover, if the ties are made too tight, they can potentially choke the patient. The cloth ties are also susceptible to bacteria, creating a greater risk of infection around the puncture area for the tube or the skin around the neck area. Finally, the cloth ties are inconvenient, requiring the clinician or care taker to dispose of the holders and reapply daily.

### SUMMARY OF THE INVENTION

The present invention alleviates to a great extent the disadvantages presented by the prior art devices by providing for a tracheostomy securement device of simple construction that offers substantial safety, easy of use, reduction of skin irritation, breakdown and potential infection.

Moreover, the invention provide an “overlap safety locking tabs system.” This system helps secure the neckflange tabs by overlapping a hook cover to prevent accidental tab release by disoriented or uncooperative patients.

Additionally, This method can reduce tracheostomy tube dislodgement and decannulation with, a potentially life threatening event, by protecting the neckflange tabs.

A primary object of the invention is to overcome the drawbacks and shortcomings accounted for above by traditional tracheostomy neckbands, and this is achieved by providing a device of the kind referenced. Which is characterized in that the fastening means comprises an adhesive element having an adhesive surface, distanced from the cannula, for attachment of the device to the skin of the neck a space being provided between the adhesive surface and the tracheostoma. By this attachment at each side of the stoma and close to the stoma the tracheal cannula is effectively anchored in the intended position and is prevented from movement in the stoma, also in case the stoma is located at a low level. The unpleasant feeling of having something that has a tight fitting around the neck is eliminated. The fixation of the cannula is more hygienic than that provided by prior art devices. Since the adhesive surface is padded from the tracheostoma neckflange the sensitive tissues around the tracheostoma are not exposed to harsh and painful treatment.

The overlap safety locking tabs system furthermore offers a way to prevent the traditional hook and loop neckflange tabs from becoming disengaged by normal accidental methods. The overlap locking system overlays on top of the hook neckflange tabs with a hook cover to prevent the accidental release of the neckflange tabs.

The objectives of the invention can be provided by a collar for attaching a tracheostomy tube having a neckflange to a patient's neck area, comprising:
a body comprising a first neck connector and an opposing second neck connector, the first neck connector is configured to stick to a first side of a patient's neck and the second neck connector is configured to stick to a second side of the patient's neck, the first neck connector and the second neck connector each having a silicon adhesive on a back side configured to stick to skin;
a body slot and tracheostomy tube opening in the body configured to allow a tracheostomy tube to slide in the body slot and be disposed within the tracheostomy tube opening so that the body is between the patient and the neckflange on the tracheostomy tube; and
a locking mechanism for locking the body to the neckflange and retain the tracheostomy tube within the tracheostomy tube opening.

Objectives of the invention can also be obtained by a method of attaching a tracheostomy tube having a neckflange to a patient comprising:
providing a collar for a tracheostomy tube, comprising:
   a body comprising a first neck connector and an opposing second neck connector, the first neck connector is configured to stick to a first side of a patient's neck and the second neck connector is configured to stick to a second side of the patient's neck, the first neck connector and the second neck connector each having a silicon adhesive on a back side configured to stick to skin;
   a body slot and tracheostomy tube opening in the body configured to allow a tracheostomy tube to slide in the body slot and be disposed within the tracheostomy tube opening so that the body is between the patient and the neckflange on the tracheostomy tube; and
   a locking mechanism for locking the body to the neckflange and retain the tracheostomy tube within the tracheostomy tube opening;
sliding the tracheostomy tube through the slot and into the tracheostomy tube opening;
locking the neckflange to the body using the locking mechanism; and
sticking the first neck connector to the first side of the patient's neck and sticking the second neck connector to the second side of the patient's neck to secure the tracheostomy tube to the patient so that the body is between the patient and the neckflange.

### BRIEF DESCRIPTION OF THE DRAWINGS

FIG. 1 illustrates a front view of a trach tube holder.

FIG. 2 illustrates a view of a trach tube holder.

FIG. 3 illustrates a back view of a trach tube holder.

FIG. 4 illustrates a view of a trach tube mounted in a trach tube holder.

FIG. 5 illustrates the Velco hooks and fabric on the collar, hook tabs and hook flaps.

### DETAILED DESCRIPTION OF THE INVENTION

The invention will be explained with reference to the attached non-limiting Figures.

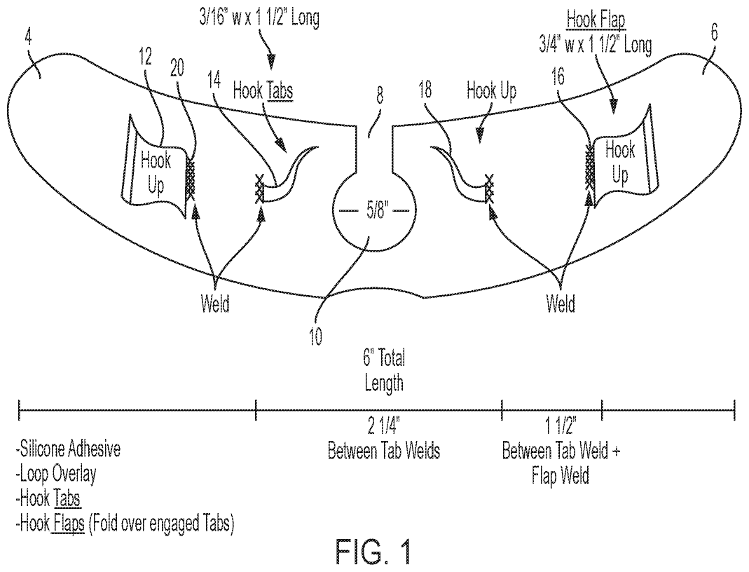

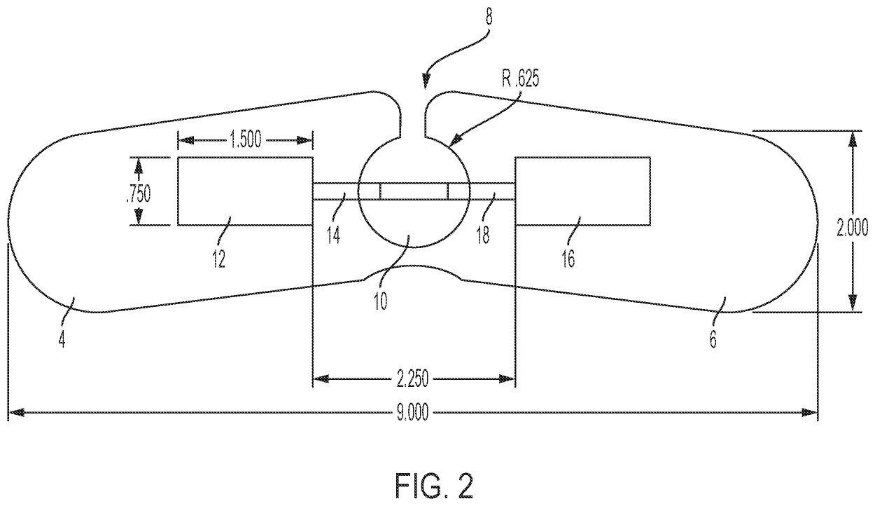

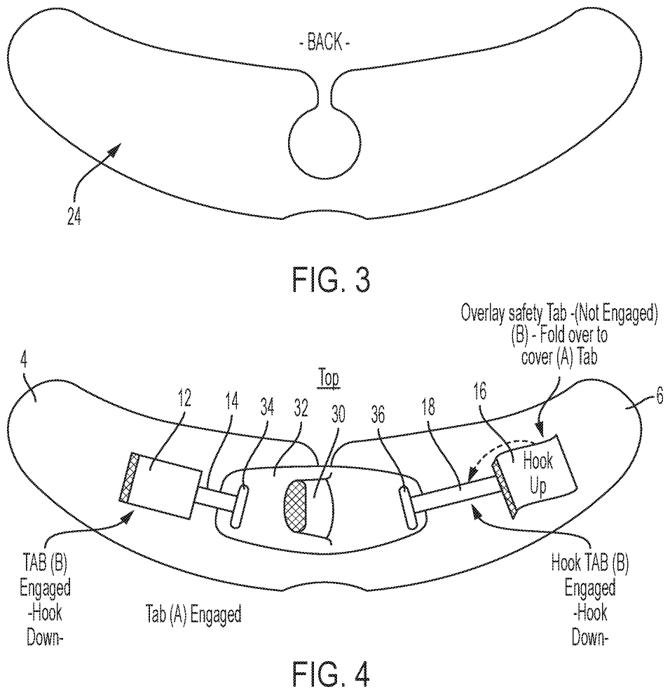

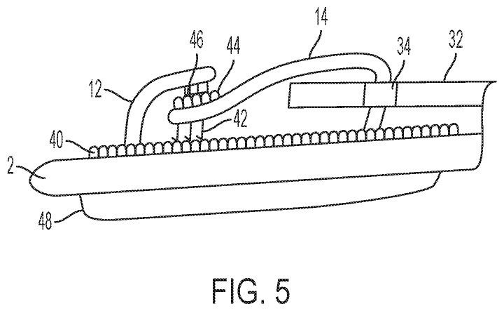

FIG. 1 illustrates a front view of a trach tube holder 2, having first neck connector 4 and a second neck connector 6 that are flexible. The neck connectors 4 and 6 are configured to attach to the sides of a patient's neck area. The backside of each neck connector 4 and 6 comprises a silicone adhesive pad 48 so that the neck connectors 4 and 6 can stick to the neck of a patient without causing irritation and allow healing. In this manner, the neck connectors 4 and 6 wings can be removed and reapplied to the patient. Silicone is the preferred adhesive pad 48. However, other suitable adhesives can be used as desired.

A typical trach tube 30 has a neckflange 32 having slots 34 and 36 for fastening the trach tube 30 to a trach tube holder 2. The body 2 comprises a locking mechanism for locking the neckflange 32 to the body 2. An example of a locking mechanism comprises hook tabs 14 and 18 that are connected to the body 2, such as by a weld 20. A front side of the body 2, opposite the back side, can comprise a body fabric material 40 that a Velcro type hook can reversibly attach to. A free end of the hook tabs 14 and 18 each comprise Velcro-type tab hooks 42 on a first side so that the hook tabs 14 and 18 can be reversibly fastened to the body fabric material 40. The body 2 also preferably comprises hook flaps 12 and 16 that are connected to the body, such as by a weld 20. A free end of the hook flaps 12 and 16 each comprises Velcro-type flap hooks 46 on a first side. A second side of the free end of the hook tabs 14 and 18, opposite the first side, each comprise a tab fabric material 44 that a Velcro hook can reversibly attach to. The flap hooks 46 on first hook flap 12 can reversibly attach to the tab fabric material 44 on the first tab 14 to further lock the first tab 14. The flap hooks on second hook flap 16 can reversibly attach to the tab fabric material on the second tab 18 to further lock the second tab 18. The hook flaps 12 and 16 can be used to lock the hook tabs 14 and 18 to prevent a patient from removing the hook tabs 14 and 18.

The invention also provides a method of attaching a tracheostomy tube 30 having a neckflange 32 to a patient comprising providing a collar for a tracheostomy tube. The collar comprises a body 2 comprising a first neck connector 4 and an opposing second neck connector 6, the first neck connector 4 is configured to stick to a first side of a patient's neck and the second neck 6 connector is configured to stick to a second side of the patient's neck, the first neck connector 4 and the second neck connector 6 each having a silicon adhesive 48 on a back side configured to stick to skin, a body slot 8 and tracheostomy tube opening 10 in the body 2 configured to allow a tracheostomy tube 30 to slide in the body slot 8 and be disposed within the tracheostomy tube opening 10 so that the body 2 is between the patient and a neckflange 32 on the tracheostomy tube, and a locking mechanism for locking the body 2 to the neckflange 32 and retain the tracheostomy tube within the tracheostomy tube opening. The method further comprises sliding the tracheostomy tube 2 through the slot 8 and into the tracheostomy tube opening 10, locking the neckflange 32 to the body 2 using the locking mechanism, and sticking the first neck connector 4 to the first side of the patient's neck and sticking the second neck connector 6 to the second side of the patient's neck to secure the tracheostomy tube 2 to the patient. The method steps can be conducted in any order.

The step of using the locking mechanism can comprise sliding the free end of the first hook tab 14 through the first flange slot 34 and locking the free end of the first hook tab 14 to the body 2, such as by the Velcro action between the hooks 42 on the free end of the first hook tab 14 and the fabric material 40 on the body 2. Similarly, the free end of the second hook tab 18 can be slid through the second flange slot 36 and locked to the body 2, such as by the Velcro action between the hooks on the free end of the second hook tab 18 and the fabric material 40 on the body 2.

The method can further comprises locking the first hook tab 14 to the body 2 with the first hook flap 12 and locking the second hook tab 18 to the body 2 with the second hook flap 16, for example by the Velcro action between the hooks 46 on a free end of the first hook flap 12 and the fabric material 44 on the free end of the first hook tab 14.

The invention provides many advantages over the prior art. The present invention can avoid the use of tracheostromy ties that encircle the patient's neck. However, if desired, tape can be used to further secure the collar to the patient's neck. An elongated silicone adhesive can be used to widen the skin surface area allowing for greater securement and patch adhesion thereby reducing the chance of decannulation. The silicone adhesive avoids back of neck skin irritation and allows for healing. Silicone offers unique skin adhesion allowing for skin to breath, reapplication, movement or replacement of collar. Padding can be provided on the collar under the neckflange 32 to protect the skin and reduce pressure sores. The overlap safety locking tabs system, the use of the hook flaps 12 and 16, reduces accidental hook tab 14, 18 disengagement that can lead to decannulation and tube extraction.

While in the foregoing, embodiments of the invention have been disclosed in considerable detail for purposes of illustration, it will be understood by those skilled in the art that many of these details may be varied without departing from the spirit and scope of the invention.

1. A collar for attaching a tracheostomy tube having a neckflange to a patient's neck area, comprising:
a body comprising a first neck connector and an opposing second neck connector, the first neck connector is configured to stick to a first side of a patient's neck and the second neck connector is configured to stick to a second side of the patient's neck, the first neck connector and the second neck connector each having an adhesive on a back side configured to stick to skin;
a body slot and tracheostomy tube opening in the body configured to allow a tracheostomy tube to slide in the body slot and be disposed within the tracheostomy tube opening so that the body is between the patient and the neckflange on the tracheostomy tube; and
a locking mechanism for locking the body to the neckflange and retain the tracheostomy tube within the tracheostomy tube opening.

2. The collar according to claim 1, wherein the locking mechanism comprises a first hook tab connected to the body and configured to slide through a first flange slot on the neckflange and then lock to the body, and a second hook tab connected to the body and configured to slide through a second flange slot on the neckflange and then lock to the body.

3. The collar according to claim 2, wherein the locking mechanism further comprises a first hook flap to lock the first hook tab to the body after being slid through the first flange slot and a second hook flap to lock the second tab to the body after being slid through the second flange slot.

4. The collar according to claim 2, wherein the locking mechanism comprises Velcro.

5. The collar according to claim 3, wherein the locking mechanism comprises Velcro.

6. The collar according to claim 1, wherein the adhesive is silicon.

7. A method of attaching a tracheostomy tube having a neckflange to a patient comprising:
providing a collar for a tracheostomy tube, comprising:
a body comprising a first neck connector and an opposing second neck connector, the first neck connector is configured to stick to a first side of a patient's neck and the second neck connector is configured to stick to a second side of the patient's neck, the first neck connector and the second neck connector each having an adhesive on a back side configured to stick to skin;
a body slot and tracheostomy tube opening in the body configured to allow a tracheostomy tube to slide in the body slot and be disposed within the tracheostomy tube opening so that the body is between the patient and the neckflange on the tracheostomy tube; and
a locking mechanism for locking the body to the neckflange and retain the tracheostomy tube within the tracheostomy tube opening;
sliding the tracheostomy tube through the slot and into the tracheostomy tube opening;
locking the neckflange to the body using the locking mechanism; and
sticking the first neck connector to the first side of the patient's neck and sticking the second neck connector to the second side of the patient's neck to secure the tracheostomy tube to the patient so that the body is between the patient and the neckflange.

8. The method according to claim 7, wherein the locking mechanism comprises a first hook tab connected to the body and configured to slide through a first flange slot on the flange a second hook tab connected to the body and configured to slide through a second flange slot on the flange, and the method comprises sliding an end of the first hook tab through the first flange slot and locking the end of the first hook tab to the body, and sliding an end of the second hook tab through the second flange slot and locking the end of the second hook tab to the body.

9. The method according to claim 8, wherein the locking mechanism further comprises a first hook flap connected to the body to lock the first hook tab to the body and a second hook flap connected to the body to lock the second hook tab to the body, and the method comprises locking the first hook tab to the body with the first hook flap and locking the second hook tab to the body with the second hook flap.

10. The method according to claim 8, wherein the locking mechanism comprises Velcro.

11. The method according to claim 9, wherein the locking mechanism comprises velcro.

12. The method according to claim 9, wherein the collar is not secured by encircling the patient's neck.

13. The method according to claim 7, wherein the adhesive is silicon.
